(19) United States
(12) Patent Application Publication
(10) Pub. No.: US 2023/0265040 A1
(43) Pub. Date: Aug. 24, 2023

(54) PROCESS FOR PURIFICATION OF PLEUROMUTILINS

(71) Applicant: Elanco Tiergesundheit AG, Basel (CH)

(72) Inventor: Larry Pierce, Basel (CH)

(21) Appl. No.: 18/140,710

(22) Filed: Apr. 28, 2023

Related U.S. Application Data
(63) Continuation of application No. PCT/US2021/057304, filed on Oct. 29, 2021.
(60) Provisional application No. 63/106,920, filed on Oct. 29, 2020.

(51) Int. Cl. C07C 68/08; C07C 69/675; C07C 323/52

(52) U.S. Cl. CPC C07C 68/08; C07C 69/675; C07C 323/52

(57) ABSTRACT

The present disclosure provides processes for making pleuromutilins.

### CROSS-REFERENCE TO RELATED APPLICATION

This application claims the benefit of U.S. Provisional Application Ser. No. 63/106,920, filed on Oct. 29, 2020, which is incorporated herein by reference.

### TECHNICAL FIELD

The present disclosure relates to medicinal chemistry, pharmacology, and veterinary and human medicine.

### BACKGROUND

Pleuromutilins are among the most modern and most effective antimicrobials currently available to veterinary medicine. Their most well-known representatives include tiamulin and valnemulin. Both substances can be very successfully used against a whole range of infectious bacterial diseases of the respiratory organs and of the digestive tract in animals.

The spectrum of activity of the pleuromutilins includes, for example, pathogens such as Streptococcus aronson, Staphylococcus aureus, Mycoplasma arthritidis, Mycoplasma bovigenitalium, Mycoplasma bovimastitidis, Mycoplasma bovirhinis, Mycoplasma sp., Mycoplasma canis, Mycoplasma felis, Mycoplasma fermentans, Mycoplasma gallinarum, Mycoplasma gallisepticum, A. granularum, Mycoplasma hominis, Mycoplasma hyorhinis, Actinobacillus laidlawii, Mycoplasma meleagridis, Mycoplasma neurolyticum, Mycoplasma pneumonia and Mycoplasma hyopneumoniae.

WO/2004/015122 A1 discloses a method for preparing one or more pleuromutilins comprising the steps of: a) culturing a pleuromutilins-producing microorganism in a liquid culture medium; and b) extracting the pleuromutilins from the unfiltered culture medium with a water immiscible organic solvent.

WO/2018/146264 A1 discloses purification methods of pleuromutilin by means of crystallisation and/or recrystallisation. The process is carried out in the presence of i-propylacetate.

### BRIEF DESCRIPTION OF THE DRAWINGS

FIG. 1 shows an HPLC analysis of Pleuromutilin starting material (content of Pleuromutilin-2,3-epoxide is about 0.3%).

FIG. 2 shows a UHPLC-MS comparison of positive AlCl₃ containing reaction mixtures after 3 h.

FIG. 3 shows a UHPLC-MS of a reference sample (no AlCl₃, no p-Toluenesulfonic acid).

FIG. 4 shows a UHPLC-MS of a reaction mixture (no AlCl₃, 10 mol % p-Toluenesulfonic acid), * indicates position of substituents is unclear.

FIG. 5 shows a UHPLC-MS of a reference sample (10 mol % AlCl₃, no p-Toluenesulfonic acid). * indicates position of substituents is unclear.

FIG. 6A shows a UHPLC-UV evaluation of the reaction product formed (HPLC-UV, Upper line: 0.5 mol % AlCl₃ & 10 mol % A p-Toluenesulfonic acid. Middle line: No AlCl₃ & 10 mol % p-Toluenesulfonic acid. Bottom line: Control, No Reaction). The main reaction product at RT 32.9 min corresponds to the diene reaction product.

FIG. 6B shows a UV spectrum of the main reaction product at RT 32.9 min (corresponds to the diene reaction product).

### DETAILED DESCRIPTION

Tiamulin, also known as [(1S,2R,3S,4S,6R,7R,8R)-4-ethenyl-3-hydroxy-2,4,7,14-tetramethyl-9-oxo-6-tricyclo[5.4.3.0^{1.8}]tetradecanyl] 2-[2-(diethylamino)ethylsulfanyl]acetate, has the structure of formula (1):

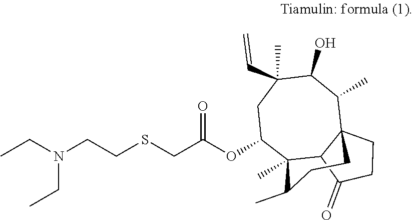 [SMILES: C=C[C@]1(C)C[C@@H](OC(=O)CSCCN(CC)CC)[C@]2(C)C(C)CC[C@]3(CCC(=O)C32)[C@@H](C)[C@@H]1O]

Tiamulin and its salts, including the hydrogen fumurate salt, are useful for the treatment and prophylaxis of a number of diseases, such as for example, dysentery, pneumonia and mycoplasmal infections in pigs and poultry.

Tiamulin and its salt forms are produced by fermentation of pleuromutilin [formula (2)], and subsequent chemical modification.

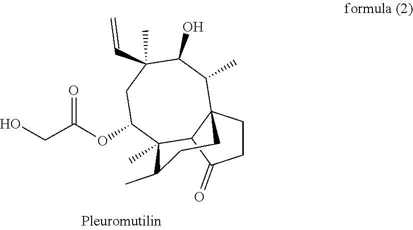 [SMILES: C=C[C@]1(C)C[C@@H](OC(=O)CO)[C@]2(C)C(C)CC[C@]3(CCC(=O)C32)[C@@H](C)[C@@H]1O]

The resultant tiamulin final product (e.g., tiamulin hydrogen fumurate) contains up to 1% of an epoxide compound of formula (4). The epoxide moiety in formula (4) permits classification of this substance as a potential genotoxic impurity (PGI).

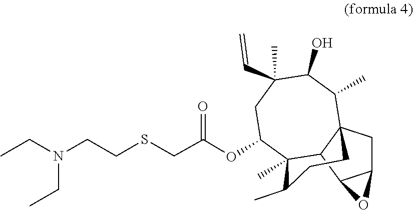 [SMILES: C=C[C@]1(C)C[C@@H](OC(=O)CSCCN(CC)CC)[C@]2(C)C(C)CC[C@]3(C[C@H]4O[C@H]4C32)[C@@H](C)[C@@H]1O]

The corresponding epoxide precursor of formula (4) is already present in pleuromulin as a fermentation by-product [formula (5)], formed at appreciable levels due to the innate biological activity of oxidation/epoxidation enzymes in the fermentation mixture.

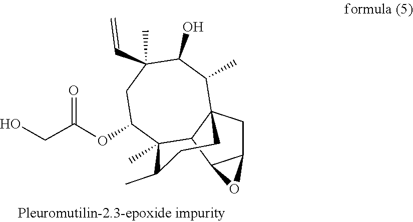 [SMILES: C=C[C@]1(C)C[C@@H](OC(=O)CO)[C@]2(C)C(C)CC[C@]3(C[C@H]4O[C@H]4C32)[C@@H](C)[C@@H]1O]

While methods of making pleuromutilins are known, there exists a need for improved methods of manufacture, and more particularly, methods that reduce undesirable epoxide impurity content in pleuromutilins to acceptable levels.

In one aspect, the present disclosure provides a method of reducing epoxide impurity content in compositions of pleuromutilin class compounds (e.g., pleuromutilin, tiamulin, valnemulin, retapamulin, lefamulin). The disclosed methods provide for reduction of epoxide impurity by 95-99% (capable of tolerating variable starting epoxide impurity content based upon stoichiometric control), thereby reducing the epoxide content in the final product to ≤0.5% (5000 ppm), preferably ≤0.1% (1000 ppm), more preferably ≤0.05% (500 ppm), even more preferably ≤0.01% (100 ppm).

In another aspect, the present disclosure provides compositions of pleuromutilin class compounds (e.g., pleuromutilin, tiamulin, valnemulin, retapamulin, lefamulin) having an epoxide impurity content of ≤0.5% (5000 ppm), preferably ≤0.1% (1000 ppm), more preferably ≤0.05% (500 ppm), even more preferably ≤0.01% (100 ppm).

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” when used in connection with a measurable numerical variable, refers to the indicated value of the variable and to all values of the variable that are within the experimental error of the indicated value or within +10 percent of the indicated value, whichever is greater.

The term, “administering to a subject” includes but is not limited to cutaneous, subcutaneous, intramuscular, mucosal, submucosal, transdermal, oral or intranasal administration. Administration could include injection or topical administration.

The term “alkyl,” as used herein, means a straight or branched, saturated hydrocarbon chain containing from 1 to 10 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond and from 1 to 10 carbon atoms.

Definitions of specific functional groups and chemical terms are described in more detail below. For purposes of this disclosure, the chemical elements are identified in accordance with the Periodic Table of the Elements. CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons. Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkenyl,” as used herein, means a straight or branched, hydrocarbon chain containing at least one carbon-carbon double bond and from 1 to 10 carbon atoms.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂.

The term “alkylamino,” as used herein, means at least one alkyl group, as defined herein, is appended to the parent molecular moiety through an amino group, as defined herein.

The term “amide,” as used herein, means —C(O)R^{x}— or —R^{x}C(O)—, wherein R^{x} may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{x}R^{y}, wherein R^{x} and R^{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NR^{x}— wherein R^{x} may be hydrogen, alkyl, cycloalkyl, aryl, heteroaryl, heterocycle, alkenyl, or heteroalkyl.

The term “aryl,” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, and tetrahydroquinolinyl.

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl,” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein, a heteroaryl group as defined herein, or a heterocycle as defined herein. Representative examples of such cycloalkyl groups include, but are not limited to, 2,3-dihydro-1H-indenyl (e.g., 2,3-dihydro-1H-inden-1-yl and 2,3-dihydro-1H-inden-2-yl), 6,7-dihydro-5H-cyclopenta[b]pyridinyl (e.g., 6,7-dihydro-5H-cyclopenta[b]pyridin-6-yl), and 5,6,7,8-tetrahydroquinolinyl (e.g., 5,6,7,8-tetrahydroquinolin-5-yl).

The term “cycloalkenyl,” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyl include, but are not limited to, alkyl ethers, secondary and tertiary alkyl amines, amides, and alkyl sulfides.

The term “heteroaryl,” as used herein, refers to an aromatic monocyclic ring or an aromatic bicyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, pyrazinyl, pyridazinyl, pyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, benzimidazolyl, benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl, quinazolinyl, 1,2,4-triazinyl, 1,3,5-triazinyl, isoquinolinyl, quinolinyl, 6,7-dihydro-1,3-benzothiazolyl, imidazo[1,2-a]pyridinyl, naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl imidazolinyl, imidazolidinyl, isothiazolinyl, isothiazolidinyl, isoxazolinyl, isoxazolidinyl, morpholinyl, oxadiazolinyl oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3,3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

In some instances, the number of carbon atoms in a hydrocarbyl substituent (e.g., alkyl or cycloalkyl) is indicated by the prefix “Cₓ-C_{y}-”, wherein x is the minimum and y is the maximum number of carbon atoms in the substituent. Thus, for example, “C₁-C₃-alkyl” refers to an alkyl substituent containing from 1 to 3 carbon atoms.

The term “sulfonamide,” as used herein, means —S(O)₂R^{d}— or —R^{d}S(O)—, wherein R^{d} may be hydrogen, alkyl, cycloalkyl, aryl, heteroaryl, heterocycle, alkenyl, or heteroalkyl.

The term “animal” is used herein to include all vertebrate animals, including humans. It also includes an individual animal in all stages of development, including embryonic and fetal stages.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “control”, “controlling” or “controlled” refers to include without limitation decreasing, reducing, or ameliorating the risk of a symptom, disorder, condition, or disease, and protecting an animal from a symptom, disorder, condition, or disease. Controlling may refer to therapeutic, prophylactic, or preventative administration. For example, a larvae or immature heartworm inflection would be controlled by acting on the larvae or immature parasite preventing the infection from progressing to an infection by mature parasites.

The term “effective amount” refers to an amount which gives the desired benefit to the subject and includes administration for both treatment and control. The amount will vary from one individual subject to another and will depend upon a number of factors, including the overall physical condition of the subject and the severity of the underlying cause of the condition to be treated, concomitant treatments, and the amount of compound of the invention used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present disclosure, the active ingredient treatment dosage, may range from, for example, 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present disclosure, the active ingredient treatment dosage, may range from, for example, 0.1 mg to 10 mg/kg of the subject. The dosing regimen is expected to be daily, weekly, or monthly administration.

The term “enantiomerically pure” refers to the (S)-enantiomer that is greater than 90%, that is, an 80% enantiomeric excess or 90% (S)-enantiomer and 10% (R)-enantiomer. In one embodiment, the term “enantiomerically pure” refers to the (S)-enantiomer that is present in greater than 92% and 8% (R)-enantiomer. In one embodiment, the term “enantiomerically pure” refers to the (S)-enantiomer that is present in greater than 94% and 6% (R)-enantiomer. In one embodiment, the term “enantiomerically pure” refers to the (S)-enantiomer that is present in greater than 96% and 4% (R)-enantiomer.

The term “salt” refers to salts of veterinary or pharmaceutically acceptable organic acids and bases or inorganic acids and bases. Such salts are well known in the art and include those described in Journal of Pharmaceutical Science, 66, 2-19 (1977). The salts may be prepared during the final isolation and purification of the compound or separately by reacting an amino group of the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compound may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine. N,N-dimethylaniline. N-methylpiperidine, N-methylmorpholine, dicyclohexylamine, procaine, dibenzylamine, N,N-dibenzylphenethylamine, 1-ephenamine and N,N-dibenzylethylenediamine, ethylenediamine, ethanolamine, diethanolamine, piperidine, piperadine, and the like.

The terms “subject” and “patient” refers includes humans and non-human mammalian animals, such as dogs, cats, mice, rats, guinea pigs, rabbits, ferrets, cows, horses, sheep, goats, and pigs. It is understood that a more particular subject is a human. Also, a more particular subject are mammalian pets or companion animals, such as dogs and cats and also mice, guinea pigs, ferrets, and rabbits.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, dialkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl. For example, if a group is described as being “optionally substituted” (such as an alkyl, alkenyl, alkynyl, aryl, heteroaryl, cycloalkyl, heteroalkyl, heterocycle or other group such as an R group), it may have 0, 1, 2, 3, 4 or 5 substituents independently selected from halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, dialkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The terms “treating”, “to treat”, “treated”, or “treatment”, include without limitation restraining, slowing, stopping, reducing, ameliorating, reversing the progression or severity of an existing symptom, or preventing a disorder, condition, or disease. For example, an adult heartworm infection would be treated by administering a compound of the invention. A treatment may be applied or administered therapeutically.

The skilled artisan will appreciate that certain of the compounds of the present invention exist as isomers. All stereoisomers of the compounds of the invention, including geometric isomers, enantiomers, and diastereomers, in any ratio, are contemplated to be within the scope of the present invention. The skilled artisan will also appreciate that certain of the compounds of the present invention exist as tautomers. All tautomeric forms the compounds of the invention are contemplated to be within the scope of the present invention.

Compounds of the invention also include all isotopic variations, in which at least one atom of the predominant atom mass is replaced by an atom having the same atomic number, but an atomic mass different from the predominant atomic mass. Use of isotopic variations (e.g., deuterium, ²H) may afford greater metabolic stability. Additionally, certain isotopic variations of the compounds of the invention may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHODS

In one aspect, the present disclosure provides a process for purifying a pleuromutilin class compound, the process entails treating a composition comprising a pleuromutilin and one or more impurities with a nucleophile to generate one or more impurity-nucleophile reaction adducts, and optionally purifying the pleuromutilin from at least one of the one or more impurity-nucleophile reaction adducts.

In certain embodiments, the pleuromutilin has formula (6), and the one or more impurities have formula (7),

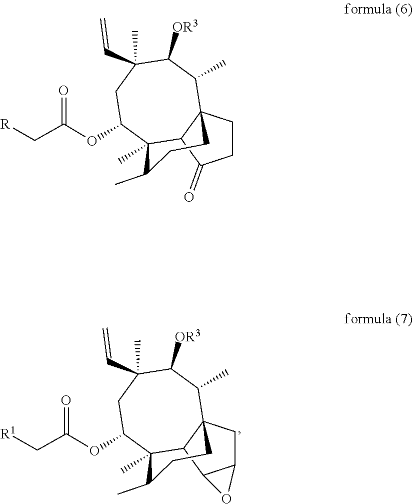

wherein
   R and R¹ are each independently selected from —OH.

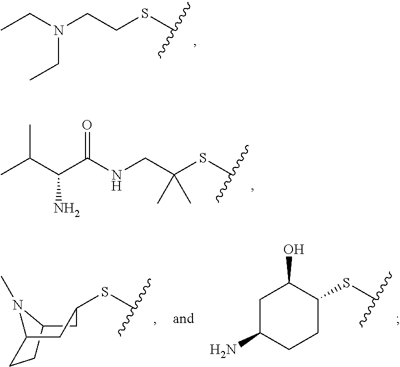

and
   R³ is H.

In certain embodiments, the nucleophile is a halogen; a carbon nucleophile; a boronic acid; an oxygen nucleophile (e.g., an alcohol, an ether, an organic acid); a nitrogen nucleophile (e.g., ammonia, an amine, an azide, a cyanide, an isocyanate, an isothiocyanate); a sulphur nucleophile (e.g., a thiol, a thioether); a selenocyanate; a phosphine, or a Grignard reagent. In a preferred embodiment, the nucleophile is an organic acid, more preferably fumaric acid or p-Toluene sulfonic acid, even more preferably p-Toluenesulfonic acid (also trivially referred to as p-TSA, p-TsOH, or tosic acid, where p=‘para’ or 4-phenyl substitution position).

In certain embodiments, the nucleophile has formula (8): R²—OH, wherein R² is selected from H, alkyl, alkenyl, alkynyl, —S(O)—R⁴, —S(O)₂—R⁴, and —C(O)—R⁵, wherein R⁴ and R⁵ are independently selected from alkyl, alkenyl, alkynyl, aryl, heteroaryl, cycloalkyl, and heterocycloalkyl, wherein said alkyl, alkenyl, aryl, heteroaryl, cycloalkyl, and heterocycloalkyl of R², R⁴, and R⁵, are each independently substituted or unsubstituted with one or more substituents.

In certain embodiments, the nucleophile has formula (8): R²—OH, wherein R² is selected from H, —C₁-C₆-alkyl, —C₁-C₆-haloalkyl, —C₂-C₆-alkenyl, —C₂-C₆-alkynyl, —S(O)—R⁴, —S(O)₂—R⁴, and —C(O)—R⁵, wherein R⁴ and R⁵ are independently selected from —C₁-C₆-alkyl, —C₂-C₆-alkenyl, —C₆-C₁₀-aryl, -5-to 10-membered heteroaryl, —C₃-C₈-cycloalkyl, and 5-to 10-membered heterocycloalkyl, each optionally substituted, valency permitting, with 1, 2, 3, 4, or 5 substitutents independently selected from —C₁-C₆-alkyl, —C₁-C₆-haloalkyl, —C₆-C₁₀-aryl, —C₃-C₈-cycloalkyl, 5-to 10-membered heteroaryl, halogen, —OR^{b}, —NR^{d}R^{c}, —COR^{b}, —CN, —CO₂R^{b}, and —CONR^{d}R^{e}, wherein R^{b}, R^{c}, R^{d}, and R^{e} are each independently selected from —H, and —C₁-C₆-alkyl.

In certain embodiments, the nucleophilic addition reaction is conducted in the presence of an activating agent (e.g., a catalyst). In certain embodiments, the nucleophilic addition reaction is conducted in the presence of a catalyst. In certain embodiments, the nucleophilic addition reaction is conducted in the presence of one or more acids or one or more bases. In a preferred embodiment, the nucleophilic addition reaction is conducted in the presence of a Lewis acid (e.g., AlCl₃, AlBr₃, ZnCl₂, FeCl₃, BF₃, SnCl₄).

In certain embodiments, the nucleophilic addition reaction is conducted in the presence of a solvent (e.g., water, esters, alkanols, halogenated hydrocarbons, ketones, ethers), preferably a polar aprotic solvent. Exemplary solvents include, but are not limited to, methanol, ethanol, n-propanol, isopropanol, n-butanol, dichloromethane, chloroform, 1,2-dichloroethane, acetone, methyl ethyl ketone, diethyl ether, tetrahydrofuran, N,N-dimethylformamide, N,N-dimethylacetamide, dimethylsulphoxide, acetonitrile, N-methylpyrrolidone, ethyl acetate, propyl acetate, isopropyl acetate, and n-butyl acetate, or any combination thereof. In certain embodiments, the solvent is ethyl acetate. In certain embodiments, the solvent is n-butyl acetate.

In certain embodiments, the nucleophilic addition reaction is conducted under phase transfer conditions. Useful phase transfer catalysts for the reaction include for example, quaternary ammonium salts, quaternary phosphonium salts, crown ethers, and polyethylene glycol and derivatives thereof. Exemplary phase transfer catalyst of quaternary ammonium salts and phosphonium salts include those of formula (R_{T})₄T⁽⁺⁾Z⁽⁻⁾, wherein each R_{T} is independently selected from C₁-C₂₅ alkyl; T is N or P; and Z is an anion. Exemplary phase transfer catalysts include tetraethylammonium chloride, tetrapropylammonium chloride, tetrabutylammonium chloride, tetrabutylammonium bromide, tetrabutylammonium bisulfate. C₁₆H₃₃N⁽⁺⁾(butyl)₃Br⁽⁻⁾, (butyl)₄N⁽⁺⁾CH₃SO₃⁽⁻⁾, (butyl)₄N⁽⁺⁾CF₃SO₃⁽⁻⁾, methyltrialkyl (C₈-C₁₀)ammonium chloride, (CH₃CH₂)₄PCl, (C₄H₉)₄PCl, (prop)₄PBr, and hexadecyltrimethylphosphonium bromide. In certain embodiments, the nucleophilic addition reaction is conducted under ion exchange conditions (e.g., nucleophiles on heterogeneous solid supports, such as for example, sulfonate ester resins).

In certain embodiments, the nucleophilic addition reaction is conducted a temperature of 20° C. to 100° C., 25° C. to 95° C., 30° C. to 90° C., 35° C. to 85° C., 40° C. to 80° C., 45° C. to 75° C., 50° C. to 70° C., or 55° C. to 65° C. In certain embodiments, the nucleophilic addition reaction is conducted at a temperature of about 60° C.

In certain embodiments, the nucleophilic addition reaction is conducted over 0.5 to 24 hours, 1 to 12 hours, or 2 to 4 hours. In certain embodiments, the nucleophilic addition reaction is conducted over 3 hours. In certain embodiments, nucleophile is charged in solution from a head tank wherein a limited dosing rate is used.

In certain embodiments, the nucleophilic addition reaction is conducted with agitation, for example, optionally using a wide range of agitator impeller speeds ensuring adequate mixing power per unit volume, heat and mass transfer rates, in the absence of excessive vortex formation. In certain embodiments, the reaction is conducted with a mixing or stirring device operating at 50 to 1000 revolutions per minute (rpm), or 700 to 900 rpm. In certain embodiments, the nucleophilic addition reaction is conducted with agitation, for example, with a mixing or stirring device operating at 800 rpm. In certain embodiments, the nucleophilic addition reaction is conducted with agitation, for example, with a mixing or stirring device operating at 20-40 rpm, such as in a large scale reaction.

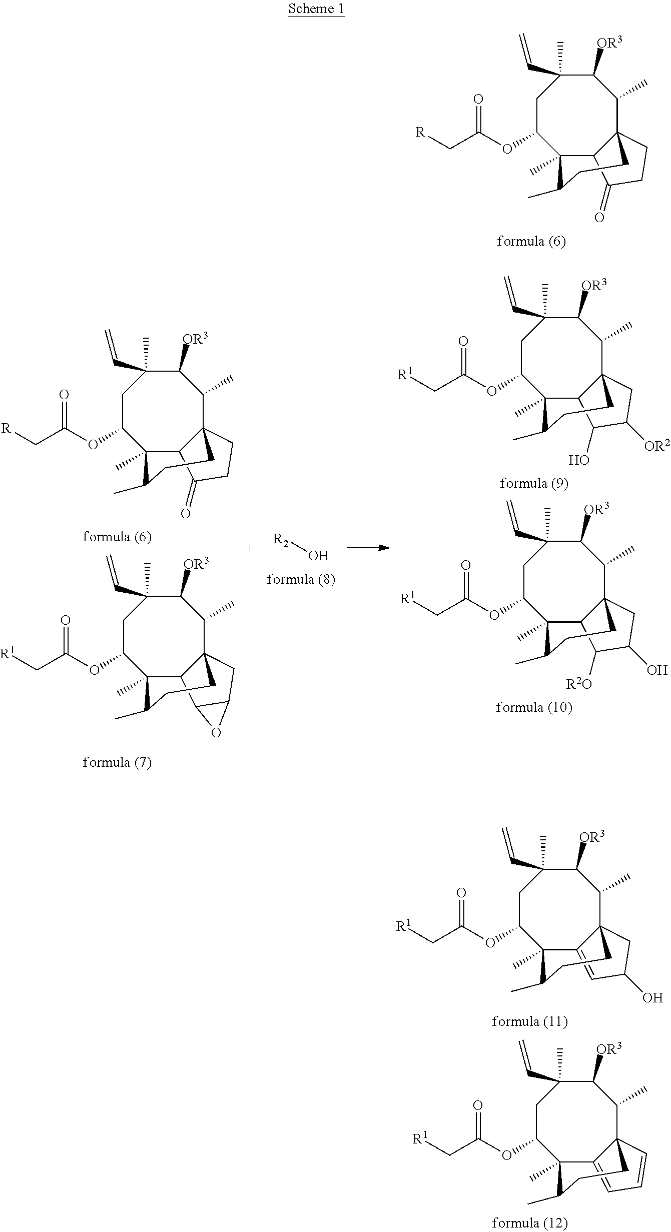

Scheme I shows an exemplary process for purifying a pleuromutilin, wherein R, R¹, R², and R³ are as defined above. The process for purifying a pleuromutilin comprises treating a composition comprising a pleuromutilin of formula (6) and an impurity of formula (7) with a nucleophile of formula (8) and optionally an activating agent, to generate a composition comprising one or more impurity-nucleophile reaction adducts. Thus, the pleuromutilin of formula (6) preferably acts as a bystander in the nucleophilic reaction between the impurity of formula (7) and the nucleophile of formula (8). In certain embodiments, at least one of the impurity-nucleophile reaction adducts has formula (9), formula (10), formula (II), or formula (12). Without wishing to be bound by theory, it is believed impurity-nucleophile reaction adducts of formula (11) and formula (12) result from one or more elimination reactions following nucleophilic addition to the epoxide of formula (7).

The pleuromutilin of formula (6) (e.g., pleuromutilin, tiamulin, valnemulin, retapamulin, lefamulin) can be purified from impurity-nucleophile reaction adducts in one or more stages in a synthetic process. For example, the initially produced impurity-nucleophile reaction adducts may be (i) purged at one or more downstream synthetic steps. (ii) carried forth as bystanders in further synthetic steps, (iii) undergo further synthetic modifications at select synthetic steps and subsequently purified away, or (iv) any combination thereof. For example, in the synthesis of tiamulin hydrogen fumurate, pleuromutilin may be treated with a nucleophile to provide a composition comprising pleuromutilin and one or more impurity-nucleophile reaction adducts. The composition comprising pleuromutilin and one or more impurity-nucleophile reaction adducts may be subjected to one or more purification processes to purify the pleuromutilin away from the impurity-nucleophile reaction adducts. Alternatively or in addition to, the composition comprising pleuromutilin and one or more impurity-nucleophile reaction adducts may be carried forth in further synthetic steps (e.g., to tiamulin or salt (thereof), and optionally the initial one or more impurity-nucleophile reaction adducts undergo synthetic modifications prior to purification away from the desired composition.

The pleuromutilin of formula (6) can be isolated and purified from at least one of the one or more impurity-nucleophile reaction adducts by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical. Essex CM20 2JE, England.

The pleuromutilin of formula (6) can in certain embodiments be isolated and purified from at least one of the one or more impurity-nucleophile reaction adducts by a process including treatment of the compositions with a base. The base may be an alkali metal hydroxide, an alkaline earth metal hydroxide, or a combination thereof. Alkali metal hydroxides include LiOH, NaOH, KOH, RbOH and CsOH. Alkaline earth metal hydroxides include Be(OH)₂, Mg(OH)₂, Ca(OH)₂, Sr(OH)₂, and Ba(OH)₂. In certain embodiments the base is KOH or NaOH.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for synthetic reactions can vary depending on the particular reactants employed and substituents present in the reactants used. Specific procedures are provided in the Examples section. Reactions can be worked up in the conventional manner (e.g. precipitation, crystallization, distillation, extraction, trituration, or chromatography). Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art: examples of which can be found in PGM Wuts and TW Greene, in Greene's book titled Protective Groups in Organic Synthesis (4^{th} ed.). John Wiley & Sons, NY (2006), which is incorporated herein by reference in its entirety.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution). Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

In another aspect, the present disclosure provides a method of purifying pleuromutilin or a salt thereof from a compound of formula (5), the method comprising (i) providing a composition comprising pleuromutilin or a salt thereof and the compound of formula (5); (ii) opening the epoxide of formula (5) with a nucleophile to provide one or more reaction adducts; and (iii) separating pleuromutilin or a salt thereof from the one or more reaction adducts.

In another aspect, the present disclosure provides a method of purifying tiamulin or a salt thereof from a compound of formula (4), the method comprising: (i) providing a composition comprising tiamulin or a salt thereof and the compound of formula (4); (ii) opening the epoxide of formula (4) with a nucleophile to provide one or more reaction adducts; and (iii) separating tiamulin or a salt thereof from the one or more reaction adducts. In certain embodiments, the tiamulin or salt thereof is tiamulin hydrogen fumurate.

In another aspect, the present disclosure provides a process for purifying a pleuromutilin class compound, the process comprising treating a composition comprising a pleuromutilin and one or more impurities with one or more reagents configured to open an epoxide functional group comprised within at least of the one or more impurities, and optionally purifying the pleuromutilin from at least one of the reaction adducts resulting from epoxide opening. In certain embodiments, the pleuromutilin class compound is pleuromutilin. In certain embodiments, the one or more impurities comprising an epoxide functional group is pleuromutilin-2,3-epoxide. In certain embodiments, the one or more reagents is p-toluenesulfonic acid or fumaric acid, optionally in the presence of a solvent (e.g., ethyl acetate or butyl acetate). In certain embodiments, the reaction is conducted at about 60° C. In certain embodiments, the pleuromutilin is purified from the reaction adducts by one or more of reaction quench (e.g., aqueous sodium hydroxide), separation (e.g., organic and aqueous layer separation), distillation, or precipitation, followed by recovery of purified pleuromutilin.

In another aspect, the process for purifying a pleuromutilin class compound, such as tiamulin, pleuromutilin, or salts thereof, occurs in the absence of crystallising and/or re-crystallising the compound, in the absence of crystallising and/or re-crystallising the compound with i-propylacetate, or in the absence of i-propylacetate.

Another aspect of the invention involves monitoring of residual nucleophiles/reagents by HPLC and removal of the residual nucleophiles/reagents by cold solvent washing of the pleuromutilin cake during isolation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. COMPOSITIONS

In one aspect, the present disclosure provides a composition comprising a pleuromutilin class compound of formula (6) substantially free of compounds of formula (7). In certain embodiments, the composition comprising a pleuromutilin class compound contains ≤0.5% (5000 ppm) of the compound of formula (7), preferably ≤0.1% (1000 ppm) of the compound of formula (7), more preferably ≤0.05% (500 ppm) of the compound of formula (7), even more preferably ≤0.01% (100 ppm) of the compound of formula (7).

In another aspect, the present disclosure provides a composition comprising pleuromutilin or a salt thereof, containing ≤0.5% (5000 ppm) of the compound of formula (5), preferably ≤0.1% (1000 ppm) of the compound of formula (5), more preferably ≤0.05% (500 ppm) of the compound of formula (5), even more preferably ≤0.01 (100 ppm) of the compound of formula (5).

In another aspect, the present disclosure provides a composition comprising tiamulin or salt thereof, containing ≤0.5% (5000 ppm) of the compound of formula (4), preferably ≤0.1% (1000 ppm) of the compound of formula (4), more preferably ≤0.05% (500 ppm) of the compound of formula (4), even more preferably ≤0.01% (100 ppm) of the compound of formula (4). Preferably the tiamulin or salt thereof is tiamulin hydrogen fumurate.

In yet another aspect, the present disclosure provides that the compositions described above comprising a pleuromutilin class compound of formula (6), pleuromutilin or a salt thereof, or tiamulin or salt thereof, may contain the epoxide impurities of compounds of formula (7), formula (5), or formula (4), respectively, in an amount less than or equal to about 0.5% (5000 ppm), 0.41 (4000 ppm), 0.3% (3000 ppm), 0.2% (2000 ppm), 0.1% (1000 ppm), 0.09% (900 ppm), 0.08% (80) ppm), 0.07% (700 ppm), 0.06% (600 ppm), 0.05% (500 ppm), 0.04% (400 ppm), 0.03% (300 ppm), 0.02% (200 ppm), or 0.01% (100 ppm). The epoxide impurity may be present in an amount greater than 0% or 0 ppm.

“Substantially free of” means containing impurities in an amount of less than or equal to about 0.5% (5000 ppm), 0.4% (4000 ppm), 0.3% (3000 ppm), 0.2% (2000 ppm), 0.1% (1000 ppm), 0.09% (900 ppm), 0.08% (800 ppm), 0.07% (700 ppm), 0.06% (600 ppm), 0.05% (500 ppm), 0.04% (400 ppm), 0.03% (300 ppm), 0.02% (200 ppm), or 0.01% (100 ppm). Impurities include, but are not limited to, the epoxide impurities of formulas (7), (5), and (4).

In yet another aspect, the present disclosure provides a purified pleuromutilin class compound of formula (6), a purified pleuromutilin or salt thereof, a purified tiamulin or salt thereof, with a purity of 99.5% or greater, 99.6% or greater, 99.7% or greater, 99.8% or greater, 99.9% or greater, 99.91% or greater, 99.92%, or greater, 99.93c or greater, 99.94% or greater, 99.95% or greater, 99.96% or greater, 99.97% or greater, 99.98% or greater, or 99.99%, or greater. The remainder percentage includes impurities, such as the epoxide impurities of formulas (7), (5), and (4), which may be present in an amount greater than 0% or 0 ppm.

In another aspect, the present disclosure provides a purified pleuromutilin composition, produced by a process that entails treating a composition comprising a pleuromutilin and one or more impurities with a nucleophile to generate one or more impurity-nucleophile reaction adducts, and purifying the pleuromutilin from at least one of the one or more impurity-nucleophile reaction adducts.

In another aspect, the present disclosure provides a purified pleuromutilin composition, produced by a process comprising (i) providing a composition comprising pleuromutilin or a salt thereof and the compound of formula (5); (ii) opening the epoxide of formula (5) with a nucleophile to provide one or more reaction adducts; and (iii) separating pleuromutilin or a salt thereof from the one or more reaction adducts.

In another aspect, the present disclosure provides a purified tiamulin composition, produced by a process comprising (i) providing a composition comprising tiamulin or a salt thereof and the compound of formula (4); (ii) opening the epoxide of formula (4) with a nucleophile to provide one or more reaction adducts; and (iii) separating tiamulin or a salt thereof from the one or more reaction adducts. In certain embodiments, the tiamulin or salt thereof is tiamulin hydrogen fumurate.

### 4. METHODS OF USE

In another aspect, disclosed are methods of treatment using the disclosed purified compositions of a pleuromutilin.

The present disclosure provides a method for the control of swine dysentery associated with Treponema hyodysenteriae susceptible to tiamulin, the method comprising administering a therapeutically effective amount of a composition comprising tiamulin or a salt thereof to a pig in need thereof, wherein the composition comprising tiamulin or a salt thereof has an epoxide impurity content [e.g., a compound of formula (4)] of ≤0.5% (5000 ppm), preferably ≤0.1% (1000 ppm), more preferably ≤0.05% (500 ppm), even more preferably ≤0.01% (100 ppm).

The present disclosure provides a method for control of porcine proliferative enteropathies (ileitis) associated with Lawsonia intracellularis, the method comprising administering a therapeutically effective amount of a composition comprising tiamulin or a salt thereof to a pig in need thereof, wherein the composition comprising tiamulin or a salt thereof has an epoxide impurity content [e.g., a compound of formula (4)] of ≤0.5% (5000 ppm), preferably ≤0.1% (1000 ppm), more preferably ≤0.05% (500 ppm), even more preferably ≤0.01% (100 ppm).

The present disclosure provides a method for the treatment of swine dysentery associated with Treponema hyodysenteriae and swine pneumonia due to Actinobacillus pleuropneumoniae susceptible to tiamulin, the method comprising administering a therapeutically effective amount of a composition comprising tiamulin or a salt thereof to a pig in need thereof, wherein the composition comprising tiamulin or a salt thereof has an epoxide impurity content [e.g., a compound of formula (4)] of ≤0.5% (5000 ppm), preferably ≤0.1% (1000 ppm), more preferably ≤0.05% (500 ppm), even more preferably ≤0.01% (100 ppm).

The present disclosure provides a method for the treatment of swine dysentery associated with Brachyspira hyodysenteriae and swine pneumonia due to Actinobacillus pleuropneumoniae susceptible to tiamulin, the method comprising administering a therapeutically effective amount of a composition comprising tiamulin or a salt thereof to a pig in need thereof, wherein the composition comprising tiamulin or a salt thereof has an epoxide impurity content [e.g., a compound of formula (4)] of ≤0.5% (5000 ppm), preferably ≤0.1% (1000 ppm), more preferably ≤0.05% (500 ppm), even more preferably ≤0.01% (100 ppm). The present disclosure provides a method for the control of swine dysentery associated with Serpulina hyodysenteriae susceptible to tiamulin and for treatment of swine bacterial enteritis caused by Escherichia coli and Salmonella choleraesuis sensitive to chlortetracycline and and treatment of bacterial pneumonia caused by Pasteurella multocida sensitive to chlortetracycline, the method comprising administering a therapeutically effective amount of a composition comprising tiamulin or a salt thereof to a pig in need thereof, wherein the composition comprising tiamulin or a salt thereof has an epoxide impurity content [e.g., a compound of formula (4)] of ≤0.5% (5000 ppm), preferably ≤0.1% (1000 ppm), more preferably ≤0.059 (500 ppm), even more preferably ≤0.01% (1) ppm).

### 5. EXAMPLES

The present invention has multiple aspects, illustrated by the following non-limiting examples.

HPLC-UV operating conditions are provided in Tables 1A and 1B.

TABLE 1A
HPLC-UV operating conditions

| | |
|---|---|
| Mobile Phase A: | Dissolve 1 mL of phosphoric acid within 2000 mL of |
| | water, mix thoroughly |
| Mobile Phase B: | Dissolve 1 mL of phosphoric acid within 200 mL of |
| | water, add 1800 mL acetonitrile, mix thoroughly |
| Column: | Agilent SB-C18, 150 × 4.6 mm, 1.8 μm |
| Flow Rate: | 1.0 mL/min |
| Detector | λ = 210 nm |
| Wavelength: | DAD Spectrum: 200-400 nm |
| Column Temp: | 50° C. |
| Injection Volume: | 5 μL |
| Run Time: | 60 min |

TABLE 1B
Gradient

| Time [min] | % A | % B |
|---|---|---|
| 0.0 | 80 | 20 |
| 25.0 | 55 | 45 |
| 50.0 | 10 | 90 |
| 52.0 | 10 | 90 |
| 52.5 | 80 | 20 |
| 60.0 | 80 | 20 |

HPLC-MS operating conditions are provided in Tables 2A, 2B, and 2C.

TABLE 2A
HPLC-MS operating conditions

| | |
|---|---|
| Mobile Phase A: | 5% acetonitrile + 0.1% formic acid |
| Mobile Phase B: | 95% acetonitrile + 0.1% formic acid |
| Column: | Agilent Zorbax Extend-C18 2.1 × 100 mm, |
| | 1.8 μm (1200 bar) |
| Flow Rate: | 0.55 mL/min |
| Detector Wavelength: | DAD Spectrum: 200-400 nm |
| Column Temp: | 50° C. |
| Injection Volume: | 1 μL |
| Run Time: | 18 min |

TABLE 2B
Gradient

| Time [min] | % A | % B |
|---|---|---|
| 0.0 | 80 | 20 |
| 1.0 | 80 | 20 |
| 8.0 | 55 | 45 |
| 13.0 | 10 | 90 |
| 15.0 | 10 | 90 |
| 15.1 | 80 | 20 |
| 18.0 | 80 | 20 |

TABLE 2C
MS Conditions

| Polarity | ES+ |
|---|---|
| Capillary (kV) | 0.80 |
| Cone (V) | 20.00 and 50.00 |
| Source Temp (° C.) | 120 |
| Probe Temp (° C.) | 600 |
| Scan (Da) | 120-1000 |

Experimental set-up for reactions are provided in Table 3. The samples are placed in 2 mL glass vials and sealed well with an aluminum crimp cap (the tightness must be tested beforehand). The samples are incubated in a mixer at 60° C., and 800 rpm (900 rpm in Example 1). The samples are taken according to specified time points and cooled down to room temperature. For HPLC and LC-MS analysis the samples are diluted with acetonitrile.

TABLE 3
Experimental Set-up

Thermomixer C-5382/928867 by Eppendorf
SmartBlock 1.5 mL-5360/J821787
Vial: crimp top, clear glass, certified, 2 mL, vial size: 12 × 32 mm (by
Agilent)
Cap: crimp, silver aluminum, PTFE/red rubber septa, 11 mm (by Agilent )

Reagents employed in the experiments are provided in Table 4.

TABLE 4
Reagents

| Name | Role |
|---|---|
| Pleuromutilin | Starting material |
| Tiamulin Base | Starting material |
| Ortho-phosphoric acid | Buffer |
| Ethyl acetate | Solvent |
| Butyl Acetate for HPLC, 99.7% | Solvent |
| Acetonitrile | Solvent |
| Water | Solvent |
| Tetrabutylammonium bisulfate puriss., ≥99.0% | Catalyst |
| Zinc chloride | Catalyst |
| N,N′-diphenylthiourea | Catalyst |
| Magnesium chloride hexahydrate | Catalyst |
| Aluminum chloride | Catalyst |
| Silver nitrate | Catalyst |
| Glycine, ACS reagent, ≥98.5% | Reactant |
| p-Toluenesulfonic acid monohydrate | Reactant |
| Formic acid | Reactant |
| Ammonia solution 25% | Reactant |
| Fumaric acid | Reactant |
| Sodium hydroxide | Reactant |
| Acetic acid glacial | Reactant |

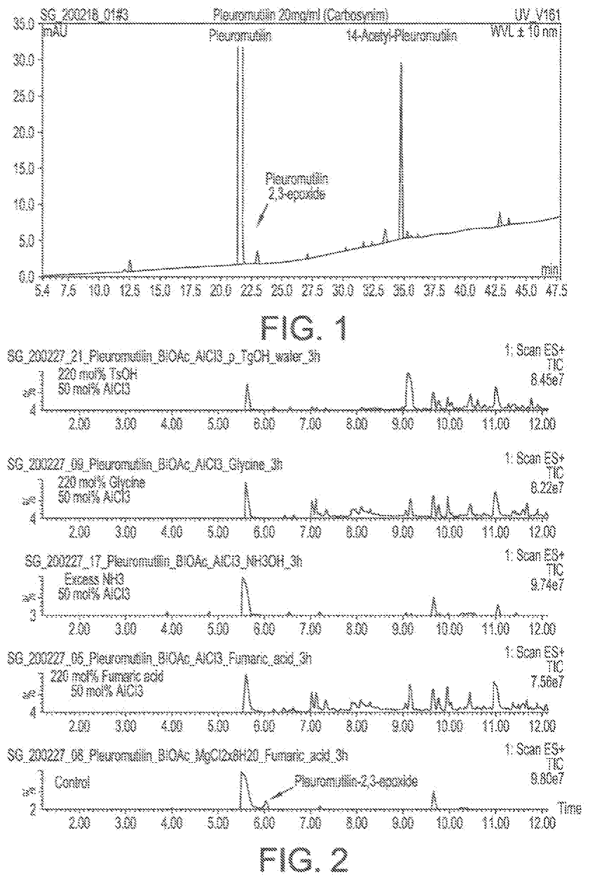

FIG. 1. HPLC of pleuromutilin starting material with epoxide impurity. The two major impurities present in the pleuromutilin sample were 14-acetyl pleuromutilin and pleuromutilin-epoxide, with the epoxide level of 0.3% being acceptably high for evaluation. Other minor impurities were also observed in the sample but were not deemed to interfere with the present work.

No reference standard for pleuromutilin-epoxide was available. Quantification via external calibration against a pleuromutilin standard was tested in the first experiment. Results showed that quantification using the peak areas of pleuromutilin-epoxide in the reaction mixtures compared to the peak areas in the negative control is completely sufficient for evaluation of the experiments. Therefore, the pleuromutilin-epoxide concentration of the negative control was defined as 100% value. %-Values given in the summary tables below refer to this value and correspond to the remaining amount of pleuromutilin-epoxide. High values indicate no depletion. Tick marks (✓) indicate complete (below detection limit) depletion of pleuromutilin-epoxide.

### Example 1

### Nucleophile Screen to Remove Epoxide Impurity

As shown in Table 5, a panel of nucleophiles along with various Lewis acid and organo-catalysts were screened to identify reagents and conditions that would lead to purification of pleuromutilin via nucleophilic addition to the pleuromutilin epoxide impurity. 4 g of a pleuromutilin stock solution was accurately weighed into a 20 mL volumetric flask. The stock solution was diluted with ethyl acetate to volume and sonicated in an ultra-sonic bath for about 1 h. The solution was milky. The catalysts and reactants were accurately weighed in 2 mL glass vials four times each. Then 1 mL of the pleuromutilin stock solution (mixed well) was added to the catalyst. This solution was added to the reactant. 16 combinations were obtained. The samples were incubated at 60° C., and 900 rpm for 3 h and 12 h. After each time point the samples were allowed to cool down. An aliquot of 50 μL was taken and diluted 1:5 with acetonitrile in a HPLC glass vial.

Negative control: 0.5 mL of the pleuromutilin stock solution was mixed with 0.5 mL of ethyl acetate.

A positive control was run to assess whether the experiment is working. For this 0.5 mL of pleuromutilin stock solution was mixed with 0.4 mL of ethyl acetate and 0.1 mL of a 0.1 N hydrochloric acid solution.

The negative and positive controls were treated like the other samples. For HPLC analysis, the negative and positive controls were diluted 1:2.5 with acetonitrile.

TABLE 5
Nucleophile Screen

| | | | | | Equivalents to PLM | | |
|---|---|---|---|---|---|---|---|
| | | | | | (approx. rel. to | | |
| Material | M.W. | Mass | | millimoles | Imp.*) | | Volume** |
| Pleuromutilin (PLM) | 378.5 | 1.0 | g | 2.6 | 1 | (300) | |
| Solvent A | | | | | | | |
| Ethyl acetate | 88.1 | | | | | | 5 mL |
| Catalyst B | | | | | | | |
| AlCl₃ | 133.3 | 0.18 | g | 1.3 | 0.5 | (150) | |
| MgCl₂•6H₂O | 203.3 | 0.27 | g | 1.3 | 0.5 | (150) | |
| ZnCl₂ | 136.3 | 0.18 | g | 1.3 | 0.5 | (150) | |
| 1,3-Diphenylthiourea | 228.3 | 0.30 | g | 1.3 | 0.5 | (150) | |
| Reactant C | | | | | | | |
| Fumaric acid | 116.1 | 0.66 | g | 5.72 | 2.2 | (660) | |
| Glycine free base | 75.1 | 0.43 | g | 5.72 | 2.2 | (660) | |
| Ammonium hydroxide | 17.0 (NH₃) | | | | | | 0.5 mL |
| p-Toluenesulfonic | 172.2 | 0.98 | g | 5.72 | 2.2 | (660) | 0.5 mL |
| acid | | | | | | | H₂O |
| (anhydrous) | | | | | | | |

*Impurity is present in the sample at about 0.3% a/a (relative to PLM)
**Actual volume was 1 ml (amounts and volumes were reduced accordingly)

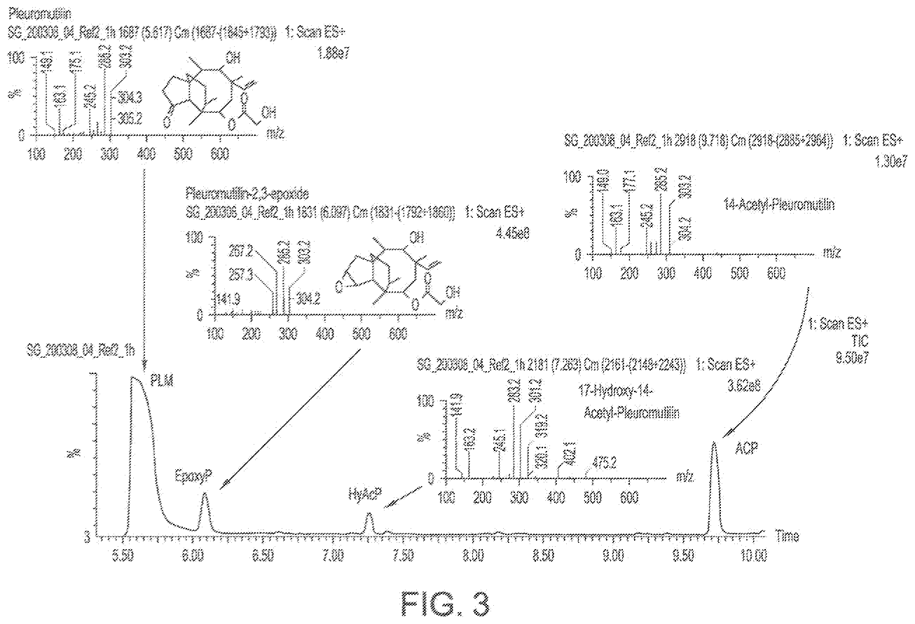

In this experiment several reactants and catalyst were screened. Concentrations of reactants and catalysts were very high to omit missing any possible reactions conditions. As shown in Table 6, a tick (✓) means that the pleuromutilin epoxide has been completely degraded (below detection limit), while a cross (x) indicates only partial or no depletion of epoxide after 3 hours. Besides the elimination of the epoxide, side reactions with pleuromutilin were visible for most of the reaction mixtures with the exception of ammonia, where only a low amount of side products were visible. See. FIG. 2.

TABLE 6
Results of Nucleophile Screen

| | | | | p-Toluenesulfonic |
|---|---|---|---|---|
| Ethyl acetate, | Fumaric | Glycine | aq. | acid/0.1 vols |
| 60° C. (3 h) | acid | free base | Ammonia | water |
| AlCl₃ | ✓ | ✓ | ✓ | ✓ |
| MgCl₂•6H₂O | x | x | x | ✓ |
| ZnCl₂ | ✓ | ✓ | x | ✓ |
| 1,3- | x | x | x | ✓ |
| Diphenylthiourea | | | | |

### Example 2

### Evaluation of p-Toluenesulfonic Acid and AlCl₃

For the pleuromutilin stock solution 3.125 g of pleuromutilin was accurately weighed into a 25 mL volumetric flask. This was diluted with ethyl acetate to volume and sonicated in an ultra-sonic bath for about 30 minutes. 1 mL of this stock solution contains 125 mg of pleuromutilin. Then all further stock solutions were prepared in 10 mL glass flasks. The catalyst stock solutions were diluted to volume with ethyl acetate, the reactant stock solutions with water. The stock solutions were sonicated in an ultra-sonic bath for about 15 min. First, 100 μL of the reactant stock solution were pipetted into 2 mL glass vials. To these solutions 100 μL of the corresponding catalyst stock solution was added. Finally, 800 μL of the pleuromutilin stock solution was added. The samples were incubated for 1 h and 3 h. After each time point the samples were allowed to cool down. Aliquots of 100 μL were taken and diluted with acetonitrile in a HPLC glass vial. The dilution was 1:2.5. Two negative controls were prepared with 0.8 mL pleuromutilin stock solution plus 100 μL ethyl acetate and 100 μL water. These samples were used as reference solutions for HPLC analysis (set to 100%). An aliquot of 100 μL was taken and diluted with acetonitrile in a HPLC glass vial. The dilution was 1:2.5.

TABLE 7
p-Toluenesulfonic acid Screen

| | | | | Stock | Stock | Ethyl | |
|---|---|---|---|---|---|---|---|
| | Mass | | | Solution | Solution | Acetate | Water |
| Material | [mg] | mMol | Equivalent | [mg/10 mL] | [μL] | [μL] | [μL] |
| Pleuromutilin | 100 | 0.264 | | | | 800 | |
| Solvent | | | | | | | |
| Ethyl acetate | | | | | | | |
| Catalyst | | | | | | | |
| No catalyst | | | | | | 100 | |
| AlCl₃ in ethyl acetate | 0.035 | 0.0003 | 0.001 | 3.5 | 100 | | |
| | 0.176 | 0.0013 | 0.005 | 18 | 100 | | |
| | 0.704 | 0.0053 | 0.02 | 70 | 100 | | |
| | 3.522 | 0.0264 | 0.1 | 352 | 100 | | |
| Reactant | | | | | | | |
| No reactant | | | | | | | 100 |
| p-Toluenesulfonic | 0.045 | 0.0003 | 0.001 | 4.5 | 100 | | |
| acid in water | | | | | | | |
| | 0.227 | 0.0013 | 0.005 | 23 | 100 | | |
| | 0.910 | 0.0053 | 0.02 | 91 | 100 | | |
| | 4.550 | 0.0264 | 0.1 | 455 | 100 | | |

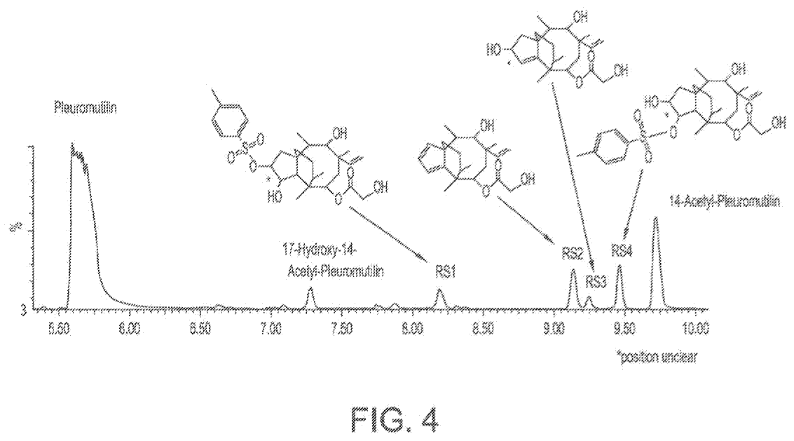

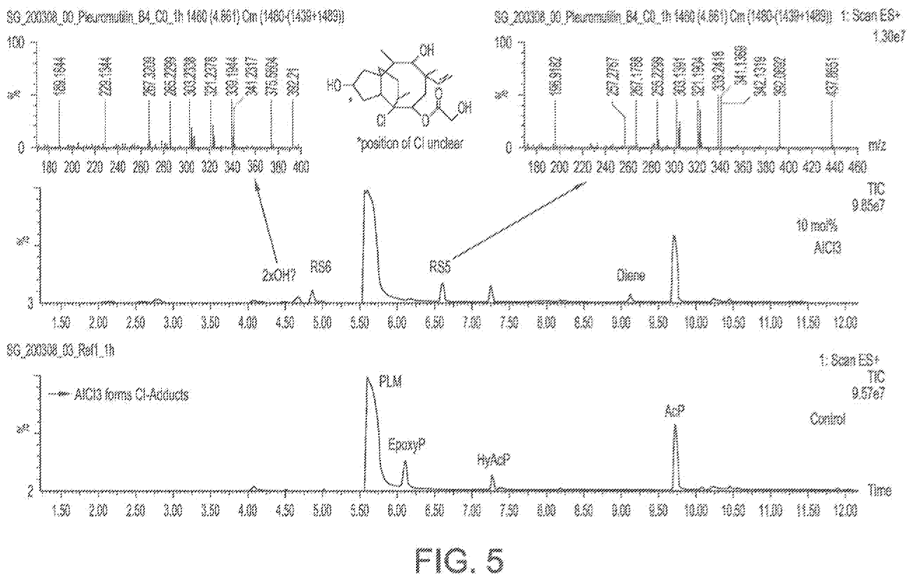

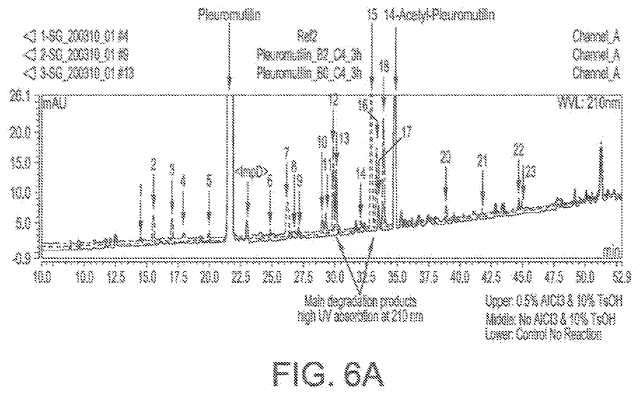

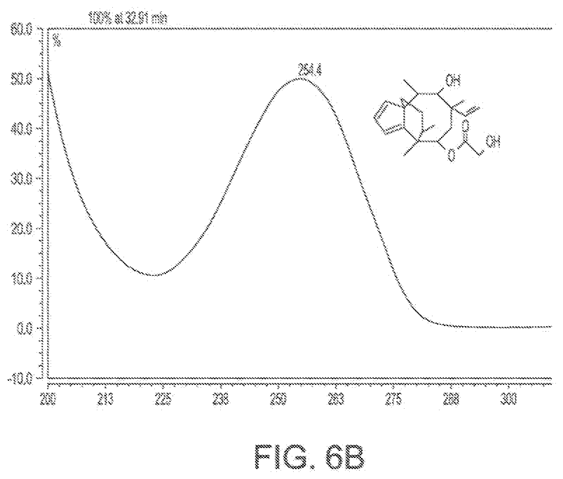

The influence of different concentration of p-Toluenesulfonic acid and AlCl₃ were studied, with results shown in Table 8. A tick (✓) means that the pleuromutilin epoxide has been completely degraded (below detection limit). As shown in FIG. 4. AlCl₃ is not required for the reaction (e.g., 0.1% mol p-Toluenesulfonic acid reduced the epoxide level to 86% after 1 hour of reaction; 0.5 mol % p-Toluenesulfonic acid reduced the epoxide level to 76% after 1 hour of reaction; and 2 mol % and 10 mol % p-Toluenesulfonic acid completely removed the epoxide after 1 hour of reaction). As shown in FIG. 5, 10 mol % AlCl₃ without p-Toluenesulfonic acid is suitable to eliminate the epoxide, via a route which produces a halogenated reaction product. Addition of a low amount of AlCl₃ and the variation of the amount of p-Toluenesulfonic acid has a strong influence on number and amount of reaction products. See FIGS. 3-6B.

TABLE 8
Results of Evaluation of p-Toluenesulfonic acid (“p-TSA”) and AlCl₃

| Ethyl Acetate | | 0.1 mol % | 0.5 mol % | 2 mol % | 10 mol % | |
|---|---|---|---|---|---|---|
| (100 mg PL/mL) | Negative | AlCl₃ | AlCl₃ | AlCl₃ | AlCl₃ | |
| 60° C. 800 rpm | control: | (=0.001 | (=0.005 | (=0.02 | (=0.1 | Time |
| (1 & 3 h) | No AlCl₃ | equiv)* | equiv)* | equiv)* | equiv)* | (h) |
| Negative | 100** | 92 | 96 | 98 | ✓ | 1 |
| control: | | 99 | 97 | 91 | ✓ | 3 |
| No p-TSA/water | | | | | | |
| 0.1 mol % | 86 | 87 | 78 | 52 | 65 | 1 |
| p-TSA/0.1 vols | 67 | 70 | 51 | 11 | 35 | 3 |
| water | | | | | | |
| 0.5 mol % p- | 76 | 61 | 34 | 20 | ✓ | 1 |
| TSA/0.1 vols | 46 | 16 | 8 | ✓ | ✓ | 3 |
| water | | | | | | |
| 2 mol % p- | ✓ | ✓ | ✓ | ✓ | ✓ | 1 |
| TSA/0.1 vols | ✓ | ✓ | ✓ | ✓ | ✓ | 3 |
| water | | | | | | |
| 10 mol % p- | ✓ | ✓ | ✓ | ✓ | ✓ | 1 |
| TSA/0.1 vols | ✓ | ✓ | ✓ | ✓ | ✓ | 3 |
| water | | | | | | |

*Relative to pleuromutilin (“PL”)
**Reference set to 100% epoxide impurity
Epoxide Impurity present in sample at about 0.3% (relative to PL)

### Example 3

### Evaluation of Butyl Acetate as a Solvent and Effect of Water

For the Pleuromutilin stock solution 5 g of Pleuromutilin was accurately weighed into a 50 mL volumetric flask. Diluted with butyl acetate to volume and sonicated in an ultra-sonic bath for about 30 minutes, 1.0 mL of this stock solution contains 10 mg of Pleuromutilin. The p-Toluenesulfonic acid stock solutions were prepared in 10 mL glass flasks. The stock solutions were diluted with butyl acetate to volume and sonicated in an ultra-sonic bath for about 15 min. 100 μL of the reactant stock solution was pipetted into 2 mL glass vials. The reactant stock solution containing 4.6 mg/mL p-Toluenesulfonic acid was pipetted twice and 100 μL of water was added to one vial. Finally, 900 μL of the Pleuromutilin stock solution was added to each vial. The samples were incubated 1 h and 3 h. The samples containing water were incubated for 3 h and 12 h. After each time point the samples were allowed to cool down. For HPLC analysis aliquots of 100 μL were taken and diluted with acetonitrile in a HPLC glass vial. The dilution was 1:2.5. Two negative controls were prepared with 0.9 mL Pleuromutilin stock solution plus 100 μL butyl acetate. These samples were used as reference solutions for the HPLC analysis (set to 100%). Aliquots of 100 μL were taken and diluted with acetonitrile in a HPLC glass vial. The dilution was 1:2.5.

TABLE 9
Change to Butyl acetate and effect of water

| | | | | Stock | | | |
|---|---|---|---|---|---|---|---|
| | Mass | | | Solution | Stock | ButAc | Water |
| Material | [mg] | mMol | Equivalent | [mg/10 mL] | [μL] | [μL] | [μL] |
| Pleuromutilin | 100 | 0.264 | | | | 900 | |
| Solvent A | | | | | | | |
| Butyl acetate | | | | | | 100 | |
| Reactant C | | | | | | | |
| p-Toluenesulfonic acid | 0.227 | 0.0013 | 0.005 | 23 | 100 | | |
| | 0.455 | 0.0026 | 0.010 | 46 | 100 | | 100 |
| | 0.455 | 0.0026 | 0.010 | 46 | 100 | | |
| | 0.910 | 0.0053 | 0.020 | 91 | 100 | | |

As shown in Table 10, the epoxide ring-opening operated effectively in butyl acetate. Additional water charging is not needed, but contribution from intrinsic residual water due to the use of non-dry solvents could not be excluded. Thus, epoxide opening with p-Toluenesulfonic acid is effective in ethyl acetate and butyl acetate (with and without water).

TABLE 10
Results of evaluation with butyl acetate and water

| Butyl acetate (100 mg | 0.1 vols | No | Time |
|---|---|---|---|
| PL/mL) 60° C. 800 rpm | water | additional water | [h] |
| 0.5 mol %* | n/a | ✓ | 1 |
| p-Toluenesulfonic acid | | ✓ | 3 |
| 1.0 mol % * | ✓ | ✓ | 1 |
| p-Toluenesulfonic acid | ✓ | ✓ | 3 |
| 2.0 mol %* | n/a | ✓ | 1 |
| p-Toluenesulfonic acid | | ✓ | 3 |

*Relative to Pleuromutilin

### Example 4

### Conditions with and without Phase Transfer Catalyst

For the pleuromutilin stock solution 5 g of pleuromutilin was accurately weighed into a 50 mL volumetric flask. Diluted with butyl acetate to volume and sonicated in an ultra-sonic bath for about 30 minutes, 1.0 mL of this stock solution contains 100 mg of Pleuromutilin. The stock solutions were prepared in 10 mL glass flasks. The stock solutions of p-Toluenesulfonic acid were diluted with butyl acetate. The other stock solutions were diluted with water to volume and sonicated in an ultra-sonic bath for about 15 min. 2 set-ups were used. In the first set-up 9 mg of Tetrabutylammonium bisulfate was accurately weighed into nine 2 ml glass vials. Then 100 μL Fumaric acid was pipetted into 3 vials and 100 μL p-Toluenesulfonic acid and 100 μL glycine into 3 more vials. No reactant was added to the remaining 3 vials. Afterwards phosphoric acid (pH 2), phosphoric acid (pH 4) and water was added to the samples. The samples were made up with 100 uL butyl acetate. Finally, each sample contained 1.0 mL butyl acetate. The samples were incubated for 3 h. The samples containing no catalyst and Fumaric acid were incubated for 1 h and 3 h. After each time point the samples were allowed to cool down. For HPLC analysis aliquots of 100 μL were taken and diluted with acetonitrile in a HPLC glass vial. The dilution was 1:2.5. The second set-up was like the first one, hut without Tetrabutylammonium bisulfate as catalyst. Two negative controls were prepared with 0.9 mL Pleuromutilin stock solution plus 100 μL butyl acetate. These samples were used as reference solutions for the H-PLC analytic (set to 100%). Aliquots of 100 μL were taken and diluted with acetonitrile in a HPLC glass vial. The dilution was 1:2.5.

TABLE 11
Conditions with and without phase transfer catalyst

| | | | | Stock | | Butyl | |
|---|---|---|---|---|---|---|---|
| | Mass | | | Solution | Stock | Acetate | Water |
| Material | [mg] | mMol | Equivalent | [mg/10 ml] | [μL] | [μL] | [μL] |
| Pleuromutilin | 100 | 0.264 | | | | 900 | |
| Solvent A | | | | | | | |
| Butyl acetate | | | | | | 100 | |
| Catalyst B | | | | | | | |
| Tetrabutyl ammonium | 8.970 | 0.0264 | 0.1 | | | | |
| bisulfate | | | | | | | |
| Reactant | | | | | | | |
| p-Toluenesulfonic | 0.910 | 0.0053 | 0.02 | 91 | 100 | | |
| acid in butyl acetate | | | | | | | |
| Glycine in H₂O | 0.595 | 0.0079 | 0.03 | 59 | 100 | | |
| Fumaric acid in H₂O | 0.613 | 0.0053 | 0.02 | 61 | 100 | 100 | |
| Phosphoric acid pH 2 | | | | | | | 400/500* |
| in water | | | | | | | |
| Phosphoric acid pH 4 | | | | | | | 400/500* |
| in water | | | | | | | |
| Water | | | | | | | 900 |

*Phosphoric acid solution of correct pH initially added (first figure) followed by water as diluent (second amount) to make it up to the correct volume (i.e. approx. 1:1 ratio of aqueous to organic phases)

Table 12 shows results of screening of other conditions in butyl acetate with and without phase transfer catalyst. All values determined Were in the range of the control without reagents. None of the conditions were effective to eliminate the epoxide impurity.

TABLE 12
Results of evaluating conditions with and without phase transfer catalyst

| Butyl acetate (100 mg/mL), | | | | | |
|---|---|---|---|---|---|
| 60° C. (3 h), 10 mol % | Aq. H₃PO₄ | Aq. H₃PO₄ | | | |
| Tetrabutylammonium | (pH 2) | (pH 2) + | Aq. H₃PO₄ | Potable | Time |
| bisulfate | No work-up | basic work-up | (pH 4) | Water | [h] |
| 2 mol % Fumaric acid | 79 | 68 | 81 | 86 | 3 |
| 2 mol %/p-Toluenesulfonic | 69 | 76 | 73 | 72 | 3 |
| acid/3 mol % glycine | | | | | |
| Control (no reagents) | 73 | 87 | 79 | 79 | 3 |
| | Aq. H₃PO₄ | Aq. H₃PO₄ | | | |
| Butyl acetate (100 mg/mL), | (pH 2) | (pH 2) + | Aq. H₃PO₄ | Potable | Time |
| 60° C. (3 h) | No work-up | basic work-up | (pH 4) | Water | [h] |
| 2 mol % Fumaric acid | 97 | | 97 | 97 | 1 |
| | 93 | 120 | 105 | 102 | 3 |
| 2 mol % p-Toluenesulfonic | 83 | 103 | 97 | 98 | 3 |
| acid/3 mol % Glycine | | | | | |
| Control (no reagents) | 90 | 110 | 103 | 105 | 3 |

### Example 5

### Evaluation of Additional Conditions, and Tests with Tiamulin Base

For the Pleuromutilin stock solution 2.5 g of Pleuromutilin was accurately weighed into a 25 mL volumetric flask, diluted with butyl acetate to volume and sonicated in an ultra-sonic bath for about 30 minutes. 1.0 mL of this stock solution contains 100 mg of Pleuromutilin. For the preparation of Tiamulin base stock solution the following procedure was performed. Tiamulin base was liquefied by heating for 60 seconds using a power setting of 600 Watts in a microwave oven. 3.25 g of liquid Tiamulin base was accurately weighed into a 25 mL flask, diluted with butyl acetate to volume and sonicated in an ultra-sonic bath for about 30 minutes. The stock solutions were prepared in 10 mL flasks. All stock solutions with exception of one of the two Fumaric acid solutions were diluted in butyl acetate. The second Fumaric acid solution was diluted with water to volume. The following samples were prepared in 2 mL glass vials. To 0.9 mL Pleuromutilin or Tiamulin base was added:
100 μL Flumaric acid in butyl acetate
100 μL Butyl acetate+100 μL Fumaric acid in water
100 μL AlCl₃+200 μL aq. ammonia
100 μL p-Toluenesulfonic acid+200 μL aq. Ammonia
100 μL Butyl acetate+430 μL 10% aq. acetic acid
100 μL Butyl acetate+430 μL aq. H₃PO₄ (pH 2)
100 μL p-Toluenesulfonic acid

The samples were incubated for 1 h and 3 h. After each time point, the samples were allowed to cool down. For HPLC analysis an aliquot of 100 μL for Pleuromutilin and an aliquot of 150 μL for Tiamulin base were taken and diluted with acetonitrile in a HPLC glass vial. The dilution for Pleuromutilin was 1:2.5 and for Tiamulin base 1:5 in each case. Two negative controls each were prepared with 0.9 mL Pleuromutilin/Tiamulin base stock solution plus 100 μL butyl acetate. These samples were used as reference solutions for HPLC analysis (set to 100%). For HPLC analysis an aliquot of 100 μL for Pleuromutilin/150 μL for Tiamulin base was taken and diluted with acetonitrile in a HPLC glass vial. The dilution for Pleuromutilin was 1:2.5 and for Tiamulin base 1:5.

TABLE 13
Evaluation of additional conditions with pleuromutilin

| | | | | Stock | | Butyl | |
|---|---|---|---|---|---|---|---|
| | Mass | | | Solution | Stock | Acetate | Water |
| Material | [mg] | mMol | Equivalent | [mg/10 ml] | [μL] | [μL] | [μL] |
| Pleuromutilin | 100 | 0.264 | | | | 900 | |
| Solvent | | | | | | | |
| Butyl acetate | | | | | | 100 | |
| Reactant | | | | | | | |
| Water | | | | | | | 100 |
| p-Toluenesulfonic acid in | 0.455 | 0.0026 | 0.01 | 46 | 100 | | |
| butyl acetate | | | | | | | |
| Fumaric acid in H₂O | 0.613 | 0.0053 | 0.02 | 61 | 100 | 100 | |
| Fumaric acid in Butyl | 0.613 | 0.0053 | 0.02 | 61 | 100 | | |
| acetate | | | | | | | |
| AlCl₃ in butyl acetate | 0.351 | 0.0026 | 0.01 | 35 | 100 | | |
| Aq. Ammonia (25%) | | | | | | | 200 |
| Phosphoric acid pH 2 in | | | | | | 100 | 430 |
| water | | | | | | | |
| 10% Acetic acid | | | | | | 100 | 430 |

TABLE 14
Evaluation of conditions with tiamulin base

| | | | | Stock | | Butyl | |
|---|---|---|---|---|---|---|---|
| | Mass | | | Solution | Stock | Acetate | Water |
| Material | [mg] | mMol | Equivalent | [mg/10 ml] | [μL] | [μL] | [μL] |
| Tiamulin base | 130 | 0.264 | | | | 900 | |
| Solvent | | | | | | | |
| Butyl acetate | | | | | | 100 | |
| Reactant | | | | | | | |
| Water | | | | | | | 100 |
| p-Toluenesulfonic acid in | 0.455 | 0.0026 | 0.01 | 46 | 100 | | |
| butyl acetate | | | | | | | |
| Fumaric acid in H₂O | 0.613 | 0.0053 | 0.02 | 61 | 100 | 100 | |
| Fumaric acid in Butyl | 0.613 | 0.0053 | 0.02 | 61 | 100 | | |
| acetate | | | | | | | |
| AlCl₃ in butyl acetate | 0.351 | 0.0026 | 0.01 | 35 | 100 | | |
| Aq. Ammonia (25%) | | | | | | | 200 |
| Phosphoric acid pH 2 in | | | | | | 100 | 430 |
| water | | | | | | | |
| 10% Acetic acid | | | | | | 100 | 430 |

As shown in Table 15, reaction with p-Toluenesulfonic acid (re-confirmed from the previous Examples) and fumaric acid in butyl acetate were determined to be effective in full depleting the pleuromutilin-2,3-epoxide impurity. Evaluation of the same reaction conditions with tiamulin base resulted in partial depletion of formula (4) under all conditions.

TABLE 15
Results of evaluation of additional conditions with pleuromutilin

| | | Result | Result | |
|---|---|---|---|---|
| | Butyl acetate (100 mg PL/mL), 60 | at 1 h | at 3 h | Summary |
| Pleuromutilin | 2 mol % Fumaric acid | ✓ | ✓ | Reaction |
| | 2 mol % Fumaric acid in water | 65 | 33 | Slow reaction |
| | 1.0 mol % AlCl₃ % 0.2 mL aq. ammonia | 78 | 75 | No Reaction |
| | 1.0 mol % p-Toluenesulfonic acid & 0.2 mL aq. ammonia | 85 | 86 | No Reaction |
| | 7:3 mixture of butyl acetate + 10% aq. Acetic acid | 91 | 87 | No Reaction |
| | 7:3 mixture of butyl acetate + H₃PO₄ (pH 2) | 89 | 84 | No Reaction |
| | 1 mol % p-Toluenesulfonic acid | ✓ | ✓ | Reaction |

As exemplified by Examples 1-5, to find suitable conditions for the depletion of Pleuromutilin-2,3-epoxide in Pleuromutilin more than 100 discrete experimental conditions were tested and evaluated by HPLC-UV and HPLC-MS. Tests were done at small scale in 2 mL glass vials with 24 reactions in parallel. Two commercially viable options were identified: Pleuromutilin-2,3-epoxide can be completely depleted in n-butyl acetate or ethyl acetate under feasible reaction conditions (time, temperature) by treatment with (i) p-Toluenesulfonic acid [e.g., 0.5-1 mol % relative to pleuromutilin (about 2-3 times excess relative to the Epoxide impurity)], or (ii) with fumaric acid [e.g., 2 mol % relative to pleuromutilin].

### Example 6

### Pleuromutilin Purification, Large Scale

Two fermentation sub-batches of mycelia each containing approximately 680 kg Pleuromutilin (equivalent amount) were mixed with ethyl acetate (extraction tank) before transfer to a second vessel where concentration to ca. 40% w/w was performed. Both sub-batches were transferred to a crystallization vessel wherein 3.3 equivalents of p-Toluenesulfonic acid were added and the mixture was stirred at 60° C., for at least 1 hour until disappearance of the Pleuromutilin epoxide. HPLC peak was confirmed by HPLC analysis. The resulting Pleuromutilin (PL) batch was then crystallized, filtered, washed, and dried under standard processing conditions.

These production scale batches were evaluated for Pleuromutilin epoxide content and, in each case, also forward processed to Tiamulin hydrogen fumurate (Tiamulin HFU) at ca. 1000 kg scale. The analytical results of both Tiamulin HFU hatches are presented in Tables 16 and 17 below.

Starting Pleuromutilin batches evaluated to contain final Pleuromutilin epoxide HPLC levels of 0.58% (lot no. PL2012046T) and 0.49% (lot no. PL2101023T) via laboratory scale purification (isolated without p-Toluenesulfonic acid treatment) were used for baseline purposes. These Pleuromutilin control samples were also converted to Tiamulin HFU under standard laboratory scale conditions, whereupon crude epoxide levels of ca. 1.1% and 0.8% were observed respectively. Final purification would not be expected to significantly alter these elevated levels and so, these results were deemed to be typical of current process capability with respect to control of Tiamulin epoxide in this manufacturing process—0.4 to 0.6% in pure Tiamulin HFU.

In the corresponding large scale batches manufactured using commercial equipment, the final Pleuromutilin epoxide levels following implementation of the p-Toluenesulfonic acid treatment were 0.15% and <0.05% (below LOD), respectively.

These Pleuromutilin batches were also sampled and use tested at laboratory scale for direct side by side comparison with the laboratory generated Tiamulin HFU control samples. The corresponding crude Tiamulin HFU epoxide levels observed were 0.2% and <0.05% respectively (c.f. 1.1% and 0.8V for the corresponding forward processed PL laboratory control samples outlined above). Crude Tiamulin HFU epoxide impurity data collected from both full production scale batches: 01982012340 (1^{st} API batch using PL lot no. PL2012046T) and 01982101204 (2nd API hatch using PL lot no. PL2101023T), were also fully comparable with these laboratory scale data (0.15% and 0.06% respectively).

The final epoxide levels in isolated, pure Tiamulin HFU were 0.10% and <0.05% respectively (see Tables 16 and 17), thereby demonstrating that this invention successfully fulfilled the primary objectives of this study without requiring any operating modifications to the ultimate Tiamulin HFU manufacturing step even at full production scale, that Pleuromutilin epoxide suppression is an effective control strategy for Tiamulin epoxide reduction, and that this p-Toluenesulfonic acid process scaled up as expected and could elegantly achieve at least 10-fold reduction in levels of the target epoxide impurity of concern.

TABLE 16
Analytical results for 1^{st} Pleuromutilin (PL) and Tiamulin HFU (API) batch

| | PL Final | | | | | | | |
|---|---|---|---|---|---|---|---|---|
| | Batch No. | Tiamulin HFU Crude | | | | Tiamulin HFU Final | | |
| | PL2012046T | | | | D(Epox.) | | | D(Epox.) |
| | PLB | Batch No. | TM | Q | & Qs | TM | Q | & Qs |
| Control - | 0.58% | HY2012046T | 96.89% | 0.52% | 1.07% | | | |
| Lab | | | | | | | | |
| Engineering - | 0.15% | 01982012340 | 97.90% | 0.06% | 0.15% | 97.7% | 0.08% | D: 0.10% |
| Batch | | | | | | | | Qs: 0.07% |
| Use test - | | HY2012046T | 98.13% | 0.06% | 0.19% | | | |
| Lab | | HY201229 | 98.25% | 0.07% | 0.17% | | | |

TABLE 17
Analytical results for 2^{nd} Pleuromutilin (PL) and Tiamulin HFU (API) batch

| | PL Final | | | | | | | |
|---|---|---|---|---|---|---|---|---|
| | Batch No. | Tiamulin HFU Crude | | | | Tiamulin HFU Final | | |
| | PL2101023T | | | | D(Epox.) | | | D(Epox.) |
| | PLB | Batch No. | TM | Q | & Qs | TM | Q | & Qs |
| Control - | 0.49% | TM2101023T | 97.89% | 0.21% | 0.78% | | | |
| Lab | | | | | | | | |
| Engineering | ND - | 2101204 | 99.21% | 0.09% | 0.06% | 99.3% | 0.07% | D: ND |
| Batch | 0.03% | | | | | | | (Below |
| | | | | | | | | reporting |
| | | | | | | | | limit- |
| | | | | | | | | 0.05%) |
| | | | | | | | | Qs: 0.10% |
| Use test - | | TM2101023T | 98.73% | ND | 0.03% | | | |
| Lab | | | | | | | | |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the al. Such changes and modifications, including without limitation those relating to the chemical structures, substituents, derivatives, intermediates, syntheses, compositions, formulations, or methods of use of the invention, may be made without departing from the spirit and scope thereof.

1. A process for purifying a pleuromutilin class compound, the process comprising treating a composition comprising a pleuromutilin and one or more impurities with a nucleophile in the presence of a solvent to generate a composition comprising pleuromutilin and one or more impurity-nucleophile reaction adducts.

2. The process of claim 1, wherein the pleuromutilin class compound is pleuromutilin.

3. The process of claim 1, wherein the one or more impurities comprise pleuromutilin-2,3-epoxide.

4. The process of claim 1, wherein the nucleophile is p-toluenesulfonic acid or fumaric acid.

5. The process of claim 1, wherein the solvent is ethyl acetate or butyl acetate.

6. The process of claim 1, wherein the process is conducted at about 60° C.

7. The process of claim 1, wherein the one or more impurity-nucleophile reaction adducts comprise at least one compound selected from the group consisting of:

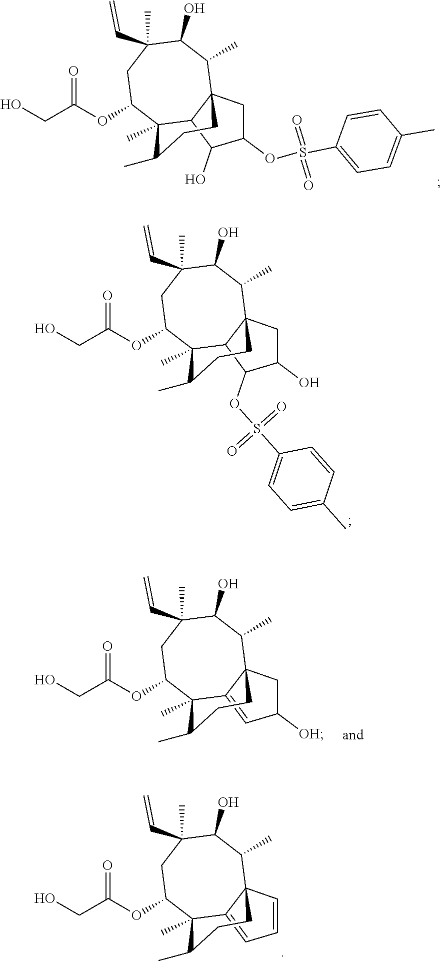 [SMILES: C=C[C@]1(C)C[C@@H](OC(=O)CO)[C@]2(C)C(C)CC[C@]3(CC(OS(=O)(=O)c4ccc(C)cc4)C(O)C32)[C@@H](C)[C@@H]1O ; C=C[C@]1(C)C[C@@H](OC(=O)CO)[C@]2(C)C(C)CC[C@]3(CC(O)C(OS(=O)(=O)c4ccc(C)cc4)C32)[C@@H](C)[C@@H]1O ; C=C[C@]1(C)C[C@@H](OC(=O)CO)[C@@]2(C)C3=CC(O)C[C@@]3(CCC2C)[C@@H](C)[C@@H]1O ; C=C[C@]1(C)C[C@@H](OC(=O)CO)[C@@]2(C)C3=CC=C[C@@]3(CCC2C)[C@@H](C)[C@@H]1O]

8. The process of claim 1, further comprising purifying pleuromutilin from at least one of the one or more impurity-nucleophile reaction adducts.

9. The process of claim 1, wherein the composition comprising a pleuromutilin and one or more impurity-nucleophile reaction adducts is treated with a base prior to isolation of the pleuromutilin class compound.

10. A composition comprising pleuromutilin or a salt thereof, containing ≤0.5% (5000 ppm) of the compound of formula (5),

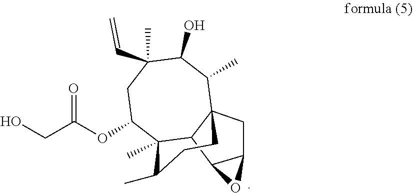 [SMILES: C=C[C@]1(C)C[C@@H](OC(=O)CO)[C@]2(C)C(C)CC[C@]3(C[C@H]4O[C@H]4C32)[C@@H](C)[C@@H]1O]

11. A composition comprising tiamulin or salt thereof, comprising ≤0.5% (5000 ppm) of the compound of formula (4),

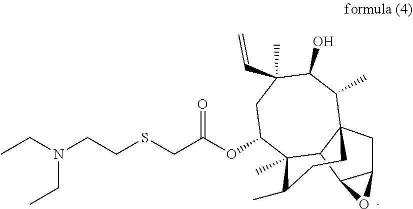 [SMILES: C=C[C@]1(C)C[C@@H](OC(=O)CSCCN(CC)CC)[C@]2(C)C(C)CC[C@]3(C[C@H]4O[C@H]4C32)[C@@H](C)[C@@H]1O]

12. The composition of claim 11, wherein the tiamulin or salt thereof is tiamulin hydrogen fumurate.

13. A composition comprising pleuromutilin and at least one compound selected from the group consisting of

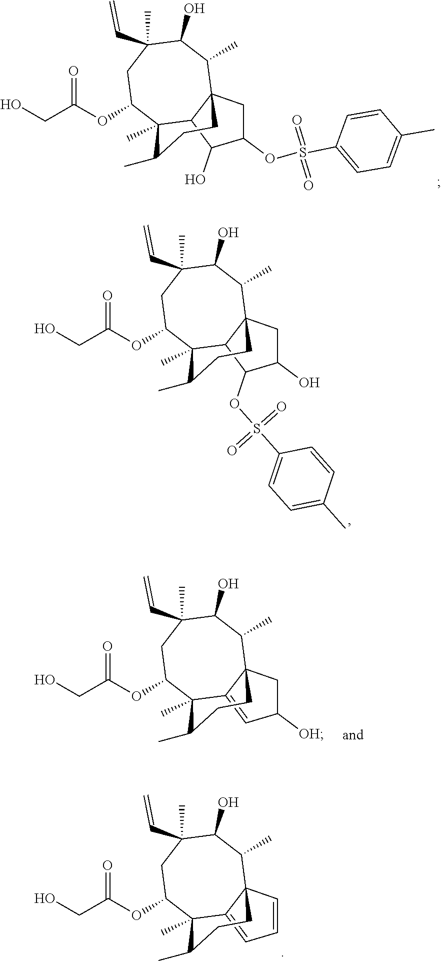 [SMILES: C=C[C@]1(C)C[C@@H](OC(=O)CO)[C@]2(C)C(C)CC[C@]3(CC(OS(=O)(=O)c4ccc(C)cc4)C(O)C32)[C@@H](C)[C@@H]1O ; C=C[C@]1(C)C[C@@H](OC(=O)CO)[C@]2(C)C(C)CC[C@]3(CC(O)C(OS(=O)(=O)c4ccc(C)cc4)C32)[C@@H](C)[C@@H]1O ; C=C[C@]1(C)C[C@@H](OC(=O)CO)[C@@]2(C)C3=CC(O)C[C@@]3(CCC2C)[C@@H](C)[C@@H]1O ; C=C[C@]1(C)C[C@@H](OC(=O)CO)[C@@]2(C)C3=CC=C[C@@]3(CCC2C)[C@@H](C)[C@@H]1O]

14. A method of purifying pleuromutilin or a salt thereof from a compound of formula (5), 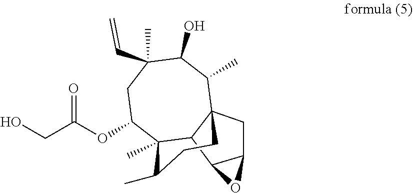 [SMILES: C=C[C@]1(C)C[C@@H](OC(=O)CO)[C@]2(C)C(C)CC[C@]3(C[C@H]4O[C@H]4C32)[C@@H](C)[C@@H]1O]
the method comprising (i) providing a composition pleuromutilin or a salt thereof and the compound of formula (5); (ii) opening the epoxide of formula (5) with a nucleophile to provide one or more reaction adducts; and (iii) separating pleuromutilin or a salt thereof from the one or more reaction adducts.

15. The method of claim 14, wherein the one or more reaction adducts have at least one compound selected from the group consisting of:

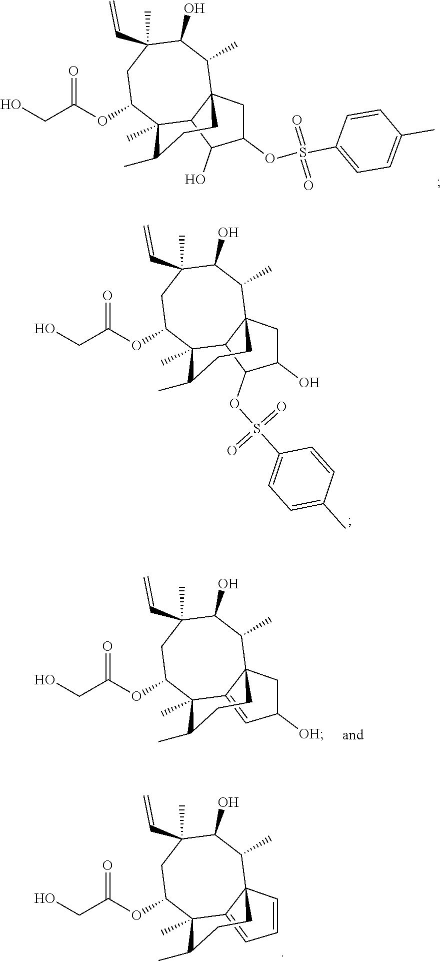 [SMILES: C=C[C@]1(C)C[C@@H](OC(=O)CO)[C@]2(C)C(C)CC[C@]3(CC(OS(=O)(=O)c4ccc(C)cc4)C(O)C32)[C@@H](C)[C@@H]1O ; C=C[C@]1(C)C[C@@H](OC(=O)CO)[C@]2(C)C(C)CC[C@]3(CC(O)C(OS(=O)(=O)c4ccc(C)cc4)C32)[C@@H](C)[C@@H]1O ; C=C[C@]1(C)C[C@@H](OC(=O)CO)[C@@]2(C)C3=CC(O)C[C@@]3(CCC2C)[C@@H](C)[C@@H]1O ; C=C[C@]1(C)C[C@@H](OC(=O)CO)[C@@]2(C)C3=CC=C[C@@]3(CCC2C)[C@@H](C)[C@@H]1O]

16. A method of purifying tiamulin or a salt thereof from a compound of formula (4), 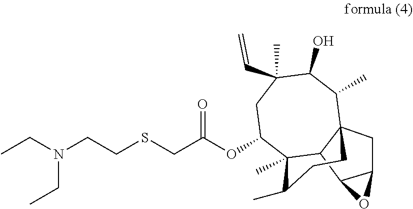 [SMILES: C=C[C@]1(C)C[C@@H](OC(=O)CSCCN(CC)CC)[C@]2(C)C(C)CC[C@]3(C[C@H]4O[C@H]4C32)[C@@H](C)[C@@H]1O]
the method comprising: (i) providing a composition comprising tiamulin or a salt thereof and the compound of formula (4); (ii) opening the epoxide of formula (4) with a nucleophile to provide one or more reaction adducts; and (iii) separating tiamulin or a salt thereof from the one or more reaction adducts.

17. The method of claim 16, wherein the tiamulin or salt therefore is tiamulin hydrogen fumurate.

18. The method of claim 14, wherein the nucleophile is p-toluenesulfonic acid or fumaric acid.

19. The method of claim 16, wherein the one or more reaction adducts have at least one compound selected from the group consisting of:

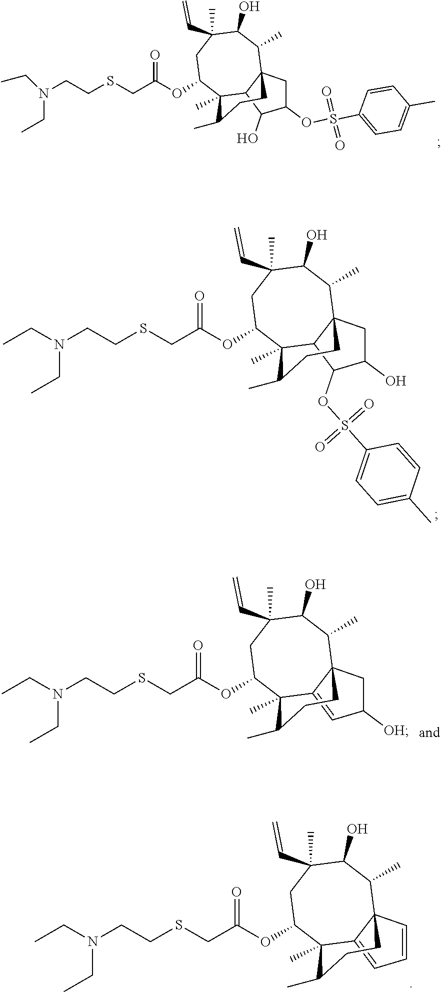

20. The method of claim 16, wherein the nucleophile is p-toluenesulfonic acid or fumaric acid.

21. The process of claim 9, wherein the base is selected from KOH and NaOH.

22. The composition of claim 10, comprising ≤0.1% (1000 ppm) of the compound of formula (5).

23. The composition of claim 10, comprising ≤0.05% (500 ppm) of the compound of formula (5).

24. The composition of claim 10, comprising ≤0.01% (100 ppm) of the compound of formula (5).

25. The composition of claim 11 comprising tiamulin or salt thereof, comprising ≤0.1% (1000 ppm) of the compound of formula (4).

26. The composition of claim 11 comprising tiamulin or salt thereof, comprising ≤0.05% (500 ppm) of the compound of formula (4).

27. The composition of claim 11 comprising tiamulin or salt thereof, comprising ≤0.01% (100 ppm) of the compound of formula (4).
